(19) United States
(12) Patent Application Publication
(10) Pub. No.: US 2022/0151684 A1
(43) Pub. Date: May 19, 2022

(54) DISPOSABLE HAND-ACTUATED ELECTROSURGICAL INSTRUMENTS

(71) Applicant: BIPAD, INC., Point Lookout, NY (US)

(72) Inventor: Louis G. Cornacchia, III, Point Lookout, NY (US)

(73) Assignee: BIPAD, INC., Point Lookout, NY (US)

(21) Appl. No.: 17/525,910

(22) Filed: Nov. 14, 2021

Related U.S. Application Data
(60) Provisional application No. 63/114,025, filed on Nov. 16, 2020.
(60) Provisional application No. 63/133,255, filed on Jan. 1, 2021.

(51) Int. Cl. A61B 18/14; A61B 18/12

(52) U.S. Cl. CPC A61B 18/1442; A61B 2018/0094; A61B 18/1206

(57) ABSTRACT

A disposable electrosurgical tool comprises a tool portion with a handle portion enabling one-handed manipulation of the tool to place an electrode at a distal end of the handle portion in contact with tissue to apply electrical current introduced to the tool portion. A switch body integrally mounted to the proximal end of the tool handle includes a push-button switch that is electrically connected to the electrode. An actuator body, removably connected to the switch body, mounts an actuator lever arm rotated by a finger of the user's hand holding the tool to close the switch. A power cord integrally connected to the switch body connects the switch to an electrical generator to introduce electrical current to the electrode when the switch is closed. The tool portion, switch body, and power cord comprise an easily sterilized, single-use, unitary electrosurgical tool.

### CROSS-REFERENCE TO RELATED PATENTS AND APPLICATIONS

This application claims the benefit of U.S. provisional application Ser. No. 63/114,025 filed Nov. 16, 2020, and U.S. provisional application No. 63/133,255 filed Jan. 1, 2021, the entire contents of both of which are incorporated by reference into the present disclosure as if set out in full. Also incorporated by reference into the present disclosure as if set out in full are U.S. Pat. No. 10,646,268 (“the '268 patent”) and International Publication No. WO 2020/154036 (“the '036 publication”), both in the name of the present inventor.

### TECHNICAL FIELD OF THE PRESENT DISCLOSURE

The present invention relates to disposable electrosurgical instruments for applying electrical current to tissue, and more particularly, to single-use instruments such as electrosurgical forceps, pencils, probes, and the like, with an integral actuator rendering the instrument capable of one-handed, multimode operation.

### BACKGROUND OF THE DISCLOSED TECHNOLOGY

The present disclosure involves improved adaptions of the hand-actuated bipolar forceps described in the '268 patent and the '036 publication. It also discloses similar hand actuation concepts for use with other types of electrosurgical instruments.

The '268 patent and '036 publication both disclose an actuator assembly adapted to be used with a conventional electrosurgical tool such as a forceps with two insulated tines having exposed electrodes at their distal tips. As seen in those documents, the tines' proximal ends are mounted to a tool plug electrically connected to the tip electrodes by conductors within the tines. FIGS. 1 and 2 of the '268 patent illustrate a conventional construction in which the forceps tool plug 16, 26 is connected to an electrical generator 60 by a separate power cord 68. A receptacle 28 at the end of the power cord has sockets 44 that accept prongs 42 on the forceps' tool plug. A surgeon articulates the forceps electrodes into position during a surgical procedure and uses a foot pedal controller 90 to close a circuit that introduces electrical current to the tool plug and thence to the forceps' tip electrodes.

The '268 patent and the '036 publication disclose numerous constructions that address the shortcomings of that arrangement. Those constructions include an actuator assembly including a switch mounted at the proximal end of the forceps and an actuating lever arm that the surgeon can manipulate with a finger to close the switch and introduce current to the electrodes once the forceps has been articulated into the desired position. FIGS. 3a and 3b of the '268 patent depict a construction with a separate actuator assembly 30 interposed between the forceps tool plug 26 and the power cord receptacle 28, with an actuating lever arm 38 pivotally mounted on the actuator assembly. The forceps plugs into the actuator assembly and the actuator assembly plugs into the power cord receptacle. That positions the lever arm so that the surgeon can operate it with a finger of the same hand holding the forceps to depress a push button switch 36 on the actuator assembly. The separate, removable actuator/switch/lever arm unit allows the surgeon to revert to the more conventional configuration in which the forceps tool plug prongs are inserted directly into the power cord receptacle sockets, with current applied via a foot pedal.

FIGS. 12-23 of the '268 patent disclose an alternate construction in which an actuator assembly 130 includes an actuating component 140 comprising a switch (like the push switch 36 in the previous embodiment), a power cord 142a, and a lever arm retaining member 150 mounted to the actuating component 140 at a hinge 148. An ergonomic lever arm 138 is removably mounted on the lever arm retaining member so that when the forceps tool plug prongs are inserted into the actuating component's receptacles (see FIG. 15), the lever arm is ideally positioned for one-finger operation by the surgeon. The lever arm is specially shaped to allow better control of switch actuation as the forceps is articulated into position (see FIGS. 16 and 17). The integrated actuating component/power cord construction has a number of advantages over the previous embodiment, a principal one being that it eliminates the need for a separate actuator assembly to convert a standard forceps to the finger operation mode that is a signature feature of both constructions.

The '036 publication discloses another advantageous construction. A preferred embodiment comprises a tool plug mount 110 with an integrated power cord 114. The tool plug prongs P1 and P2 of a conventional forceps plug into sockets 110a and 110b of the tool plug mount (see FIG. 4). The tool plug mount has a push button switch actuated by an actuating pivot arm 240 that carries an adjustable actuator lever arm 300. When the forceps tool plug TP is plugged into the tool plug mount 110, the lever arm is positioned for one-handed operation by the surgeon in the same fashion as already discussed (see FIG. 9). This construction has numerous features that increase its utility. The actuating pivot arm 240, which carries the lever arm 300, is part of an actuator body 200 that is removably attached to the tool plug mount 110 (see FIG. 3). When the actuator body is removed from the tool plug mount, the resulting forceps/plug mount combination has essentially the same “feel” as a forceps attached by its tool plug to a prior art power cord receptacle 28 (see FIG. 1 of the '268 patent). In addition, removing the actuator body is readily performed even during a surgical procedure simply by sliding it from the plug mount. Finally, converting between left- and right-hand operation is conveniently performed by unplugging the forceps from the tool plug and rotating the forceps 180° around its longitudinal axis (compare FIGS. 9 and 13). The ergonomic attributes of the construction discussed just above, which involved changing the orientation of the lever arm for right- vs. left-handed operation, are retained in the configuration in the '036 publication by employing a lever arm that can be deformed into an orientation that a surgeon, regardless of handedness, will find most convenient (see FIGS. 10-13).

### IMPROVED HAND-OPERATED ELECTROSURGICAL INSTRUMENTS

### Summary

In most surgical settings the trend is toward the use of disposable instruments to the greatest extent possible. The principal advantage is that one-use instruments avoid the necessity of sterilization after use, since the construction of many instruments makes complete sterilization difficult. In addition, an instrument designed to be sterilized multiple times often must be made more robust to be able to withstand repeated exposure to the harsh environment involved in sterilization. Electrosurgical instruments are beginning to conform to this trend, as illustrated by the known disposable forceps depicted in FIG. 1.

This figure shows a disposable bipolar forceps with tines T1 and T2 that terminate at a distal end in electrodes E1 and E2. The forceps has handle surfaces HP used by the surgeon to articulate the electrodes into the desired position at the target tissue. The proximal end of the forceps tines T1 and T2 are connected directly to an integral power cord PC, which can be connected to an electrical generator as shown in FIG. 1 of the '268 patent. The connection between the tines and the power cord is sealed in a molded end piece EP to provide a single unitary construction. Although this construction is inexpensive and readily sterilized, it can only be used for foot pedal operation like the prior art forceps having a separate power cord receptacle as shown in FIG. 1 of the '268 patent.

FIGS. 2 and 3 show other types of existing disposable bipolar instruments that connect via a separate power cord to an electrical generator in the fashion illustrated by the prior art shown in FIG. 1 of the '268 patent. That is, they not only require a separate power cord, but are also subject to many of the operational shortcomings as a disposable forceps such as that shown in FIG. 1. Further constructional details of these prior art instruments are described below.

The present disclosure relates to disposable, hand-actuated instruments, particularly to bipolar forceps and other instruments, that can realize the many advantages of a finger-operated actuating mechanism as described above and in the applicant's '268 patent and '036 publication.

### BRIEF DESCRIPTION OF THE DRAWINGS

The detailed description that follows below will be better understood when taken in conjunction with the accompanying drawings. Generally, like numerals and letters refer to like features throughout, although in some instances certain features that may have slightly different constructions, but find direct functional counterparts in different depictions, may use the same numeral/letter reference. The following is a brief identification of the drawing figures used in the detailed description.

FIG. 1 depicts a prior art disposable forceps with an integral power cord.

FIG. 2 is a perspective view of a disposable bipolar electrosurgical pencil of the type sold by Kirwan® Surgical Products LLC of Marshfield, Mass.

FIG. 3 is a perspective view of a bipolar electrosurgical probe of the type also sold by Kirwan.

FIG. 4 is a perspective view of a disposable, hand-actuated bipolar electrosurgical forceps integrated into a unitary construction comprising the forceps, a bilateral switch body, and a power cord, in accordance with an embodiment of the invention capable of right- or left-handed operation by a removable actuator body.

FIG. 5 is an exploded perspective view of the disposable forceps depicted in FIG. 4 showing further details of the switch body and the separate actuator body depicted in FIG. 4.

FIG. 6 is an exploded perspective view of a disposable, hand-actuated bipolar pencil of the type shown in FIG. 2 integrated into a unitary construction comprising the pencil, a unilateral switch body, and a power cord, with an actuator body removably mountable to the switch body.

FIG. 7 is an exploded perspective view of a disposable, hand-actuated bipolar probe of the type shown in FIG. 3 integrated into a unitary construction comprising the probe, a bilateral switch body, and a power cord, with an actuator body removably mountable to the switch body in either a right- or left-hand orientation.

One skilled in the art will readily understand that the drawings are not strictly to scale and are generally schematic in nature, but nevertheless will find them sufficient, when taken with the detailed description that follows, to make and use the devices and practice the methods described herein.

### DETAILED DESCRIPTION OF PREFERRED EMBODIMENTS

Embodiments are described more fully below in sufficient detail to enable those skilled in the art to use the described medical instruments and methods. However, embodiments may be implemented in many different forms and should not be construed as being limited to the embodiments set forth herein. The following detailed description is, therefore, not to be taken in a limiting sense. This description is intended to provide specific examples of particular embodiments illustrating various ways of implementing the subject matter described and claimed herein. It is written to take into account the level of knowledge of one of ordinary skill in the art to which such subject matter pertains. Accordingly, certain details may be omitted as being unnecessary for enabling such a person to realize the embodiments described herein.

In addition, terms used throughout are meant to have the ordinary and customary meaning that would be ascribed to them by one skilled in the art of surgery and surgical instruments. However, some of the terms used in the description herein may be explicitly defined and that definition is meant to apply throughout. When elements are referred to as being “connected” or “coupled,” the elements can be directly connected or coupled together or one or more intervening elements may also be present. In contrast, when elements are referred to as being “directly connected” or “directly coupled” there are no intervening elements present. The terms “integrated with,” “integrally mounted,” “unitary construction,” and the like, when used to refer to two or more parts, means that they are constructed as a single unit and are not separable or intended to be separated from each other.

1. Bipolar Forceps with Integrated Switch Body

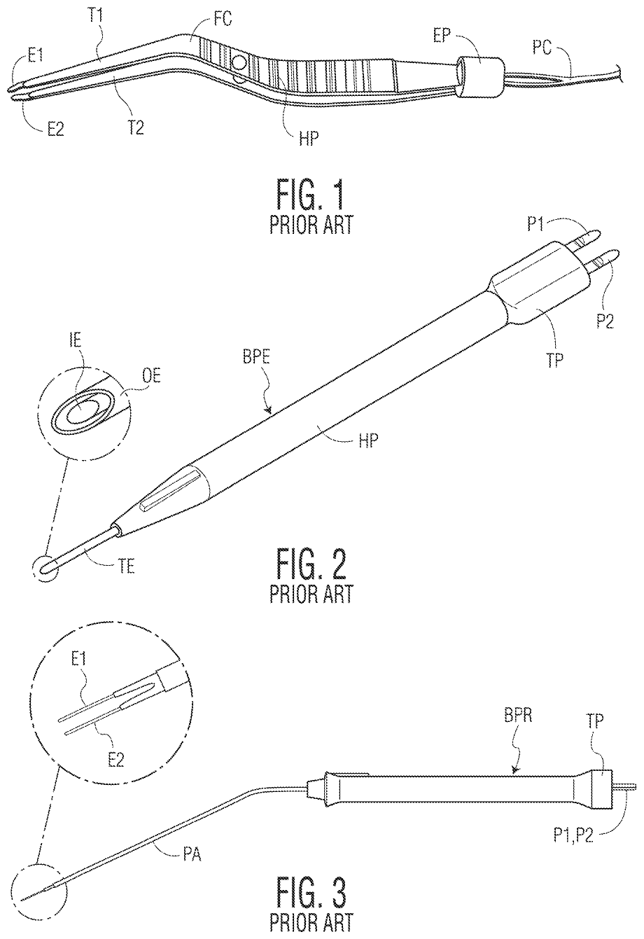

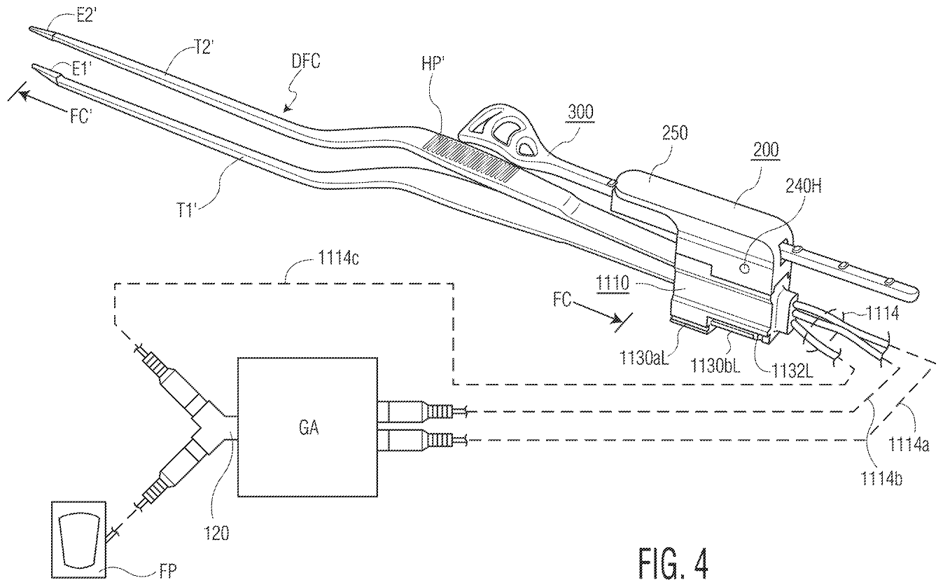

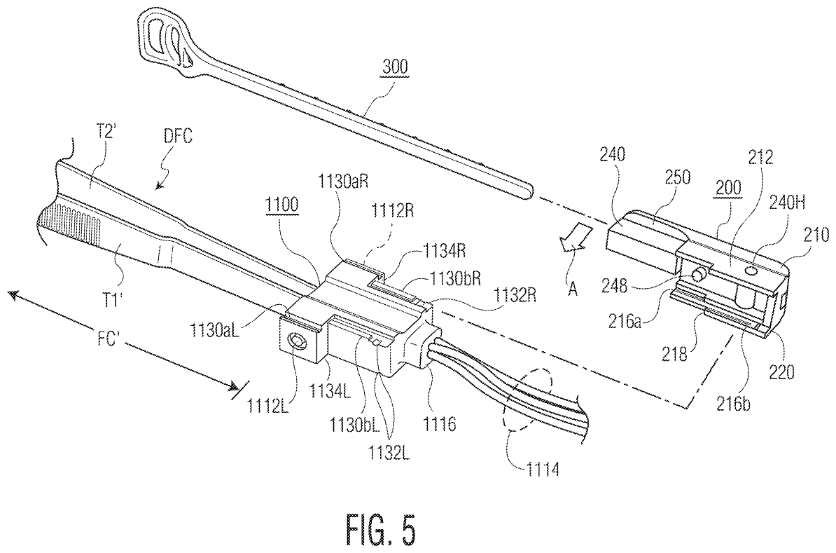

Many parts of the disposable hand-actuated forceps DFC depicted in FIGS. 4 and 5 find direct counterparts in the removable actuator assembly in the '036 publication. The same alpha and numeric designations are used in FIGS. 3 and 4 to denote corresponding parts or features in the '036 publication. This description of the disposable forceps DFC does not cover features that are already described in detail in the '036 publication and are not required for understanding the structure and operation particular to the disposable forceps that is the subject of the present disclosure. Detailed descriptions of features labeled in FIGS. 4 and 5 without an accompanying description herein can be found in the '036 publication.

The basic construction of the actual tool portion FC′ of the novel disposable forceps DFC described herein and shown in FIGS. 4 and 5 is the same as in the '036 publication, but denoted herein by a prime (′). That is, the tool portion FC′ includes a first, left tine T1′ and second, right tine T2′, which terminate at distal electrodes E1′ and E2′. The tines include a handle portion HP′ by which the user articulates the electrodes into position in contact with the target tissue. The actuator body 200 and actuator lever arm 300 are substantially identical in construction and function to those parts as described in the '036 publication.

A principal difference between the construction depicted in the drawings of the '036 publication and the novel disposable forceps disclosed herein is that the tool portion FC′ of the forceps in FIGS. 4 and 5 is integrally mounted to a bilateral switch body 1100 instead of to a tool plug. The proximal end of the forceps tool portion FC is directly coupled to and integrated with the switch body 1100, such as by molding the proximal ends of the tines into the switch body housing at the time it is formed to protect the internal components of the switch body during sterilization. The switch body in effect replaces the molded end piece EP of the prior art disposable bipolar forceps depicted in FIG. 1. The bilateral switch body is also integrated with a power cord 1114 in a manner described in more detail just below, thereby forming an integrated forceps/switch body/power cord of unitary construction. To facilitate further understanding of the construction described in the present document, parts and features that perform similarly to parts and features described in the '036 publication use similar references to which “1000” has been added. For example, the bilateral switch body identified by reference 1100 generally corresponds in certain aspects to the plug mount 100 described in the '036 publication.

FIG. 5 shows additional details of the bilateral switch body 1100. Similar to the plug mount 100 the '036 publication, the bilateral switch body 1100 includes a power cord 1114 with leads integrated with the bilateral switch body 1100 in a suitable manner. In one implementation the switch body can have a housing molded from a suitable polymeric material with a molded sealing collar 1116 that captures the leads and holds them securely in place sealed to the switch body 1110 to maintain the integrity of the seal during sterilization. The respective leads 1114a, 1114b, and 1114c of the power cord 1114 cooperate with an electrical generator GA to perform the same functions and provide the same operational features as the leads 114a, 114b, and 114c described in paragraph 0047 of the '036 publication. In particular, the present embodiment is configured for use with a Codman® Malis® CDC® III or IV bipolar electrosurgical generator, wherein the leads 1114a and 1114b are power leads and the lead 1114c is a control lead that connects to a Y-connector 120 at the generating apparatus GA to allow for selective hand operation or operation via the foot pedal FP, as described in paragraph 0040 of the '036 publication. However, the disposable forceps can be used with any other type of electrical generator that suits the purpose. For example, the Valleylab™ Force FX™ generator sold by Medtronic plc. has power leads and a control cord that terminate at a specially constructed, unitary three-prong plug, two of which carry electrical current to the forceps in response to a control input on the third responsive to foot pedal operation. In an embodiment of the disposable forceps described herein, the lead from the switch body 1110 carrying the control signal would include a separate branch for connection to an auxiliary input on the generator to permit hand control in parallel with foot pedal control in similar fashion to the embodiment in FIGS. 4 and 5.

FIGS. 4 and 5 illustrate details of the bilateral switch body 1100 specific to the present embodiment of the disposable, hand-actuated forceps DFC. The bilateral switch body 1110 includes connecting structure for removably mounting the actuator body 200. In the present embodiment, the switch body 1110 includes right- and left-hand connecting structure, denoted by the suffixes R and L in FIGS. 4 and 5, that accepts cooperating connecting structure of the actuator body 200, as described just below. To that end, the switch body 1110 connecting structure comprises ridges 1130aR, 1130bR and 1130aL, 1130bL. Female detents 1132R, 1132L are provided at the proximate ends of the corresponding ridges 1130bR, 1130bL. The ridges 1130aR and 1130bR are separated by a shoulder 1134R, and the ridges 1130aL and 1130bL are separated by a shoulder 1134L. (The switch body connecting structure at both sides is essentially basically the same in form and function as the connecting structure of the plug mount 100 shown in FIG. 4 of the '036 publication, although other constructions can be used to the same effect.).

The cooperating actuator body 200 connecting structure is identical to that shown in FIG. 4 and described in paragraph 0041 of the '036 publication. The actuator body comprises an actuator housing 210 preferably molded as a single piece. Grooves 216a and 216b molded into the internal surfaces of the housing side walls 212 accept the ridges 1130aR and 1130bR, and the ridges 1130aL and 1130bL, to provide connecting structure that permits a user to slide the actuator body onto and off of either side of the bilateral switch body 1100. Shoulders 218 separate the grooves 216a and 216b and cooperate with the shoulders 1134R and 1134L on the switch body 1100 to form a stop that positions the actuator body 200 on the switch body 1100 with their proximal and distal ends flush, as shown in the assembled view in FIG. 4. A raised male detent 220 proximate to the end of each groove 216a is accepted into the cooperating female detents 1132R and 1132L on the switch body 1100 to provide a positive “click” indication to the user that the actuator body is properly seated on the switch body 1110 and to prevent inadvertent separation of these parts while the forceps is being used.

The connecting structure for removably mounting the actuator body can take other forms besides that described. For example, in one alternate construction the connecting structure could comprise ridges molded on the actuator housing with the cooperating grooves provided in the switch body. In another construction the actuator body side walls could be made sufficiently flexible to permit the actuator body to snap onto the switch body from the side (from the viewpoint of FIG. 5). Those skilled in the art will recognize many other constructions that can accomplish the purpose of removably securing the actuator body to the switch body.

The bilateral switch body 1100 further includes an internal switch that comprises switch contacts within the switch body 1100, and a spring-biased right-hand push-button actuator 1112R and a similar spring-biased left-hand push-button actuator 1112L. The push buttons selectively place the switch contacts in the switch body in an open position in which they are not in electrical contact and a closed position in which current is conducted between the contacts. (Only the left-hand actuator 1112L is visible in FIG. B; the right-hand actuator 1112R is positioned similarly on the other side of the switch body as suggested by the dotted lead line in FIG. 5.) The push button actuators are sealed by diaphragms of a suitable flexible material to protect the internal electrical and mechanical operating components within the switch body during sterilization. The switch is in an electrical circuit between the power cord 1114 and the tool electrodes E1 and E2, whereby depressing either push-button actuator 1112R or 1112L against its spring bias electrically connects the electrical generating apparatus GA to the electrodes E1 and E2. As with the actuator assembly 10 in the '036 publication, the novel disposable forceps DFC also enables either foot pedal operation or operation at the discretion of the user.

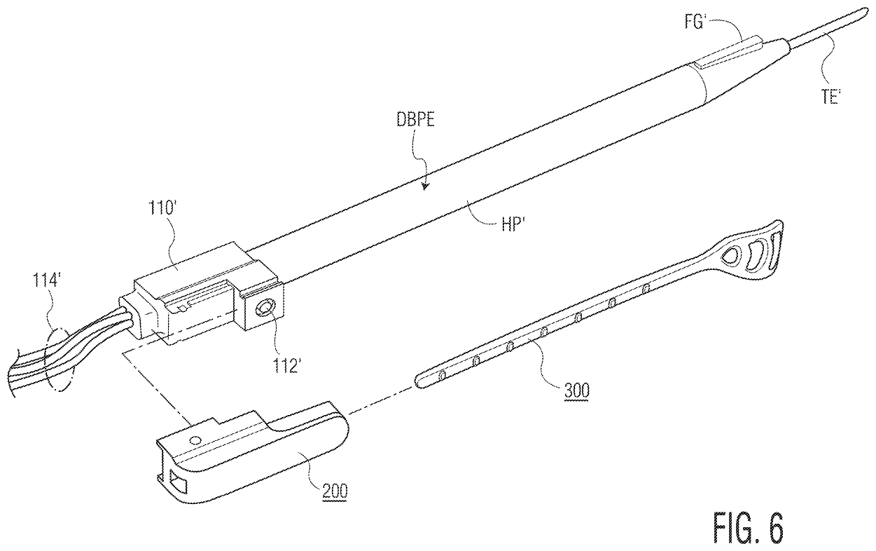

The actuator body 200 also comprises an actuating pivot arm 240, further details of which are described in paragraph 0043 of the '036 publication in connection with its FIGS. 5 and 6. The pivot arm 240 acts as a switch actuating member by rotation about the hinge point 240H to bring an actuating button 248 on the pivot arm into contact with the push-button actuator 1112R when the actuator body 200 is mounted on the switch body 1110 for right-hand operation as seen in FIG. 4. For left-hand operation, the actuator body is mounted on the other side of the switch body via the grooves 216a, 216b and the ridges 1130aL, 1130bL. The shoulders 1134R and 1134L on the switch body 1100 cooperate to place the actuating button 248 into juxtaposition with the facing switch's push-button actuator whereby rotation of the pivot arm 240 in the direction of the arrow A in FIG. 5 will depress the push button and close the switch. A projecting hood 250 extends the top wall of the actuator housing to guard against inadvertent movement of the pivot arm 240 as the forceps is manipulated by the user. This feature is described in more detail in the '036 publication in connection with FIG. 9 and the accompanying explanation in paragraph 0048.

FIG. 4 illustrates the actuator body mounted for right-handed operation as described in the '036 publication, particularly in connection with FIGS. 9-12. The actuator body 200 can also be mounted for left-handed operation as described above and shown in FIG. 13 of the '036 publication. In both orientations, the construction of the switch body 1100 and the actuator body 200 mounts the actuator lever arm 300 so that it extends toward the forceps' distal end generally along a longitude axis so that the actuator lever arm is in position for movement by a finger of the user's hand holding the forceps to close the switch by depressing the switch push button actuator 1112. (See FIGS. 11-13 of the '036 publication.)

It is anticipated that the integrated forceps/switch body/power cord unit described herein can be manufactured at a sufficiently low cost that it can be discarded after a single use, thus avoiding potential sterilization issues presented by the switch body due to its internal circuitry and switching mechanism. The actuator body and lever arm are relatively simple in configuration and can be made without areas that present sterilization challenges. Actuator body/lever arm assemblies can be maintained in inventory for repeated use with each new disposable forceps/switch/power cord unit.

Although the present description is directed to a bilateral switch body capable of use in either a right- and left-hand version, an alternate construction could comprise a unilateral switch body configured only for right- or left-hand operation. For example, in a right-hand only construction the left-hand connecting structure would be omitted from the switch body, and vice-versa for a left-hand only configuration. In another variation, the actuator body 200 can be integrated with the switch body 1100 in either the right-hand or left-hand only configuration. In other words, the instrument would have either a right-hand actuator body disposed for movement by a finger of the user manipulating a right-hand instrument or a left-hand actuator body disposed for movement by a finger of the user manipulating a left-hand instrument. In that case the entire unit would be disposable, thus avoiding the necessity for separately sterilizing the actuator bodies.

Other variations are also possible within the scope of the present description and the appended claims. For example, the actuator body 200 can accept a pistol-type actuator arm rather than the actuator lever arm 300. In that construction, the user can manipulate the forceps into position with one hand and squeeze the pistol “trigger” with the same hand to close the push button switch in the switch body 1110. In another variation the pistol grip and switch body can be integrated in the fashion described in the previous paragraph vis-à-vis the actuator body 200 and switch body. A pistol-type actuator arm will preferable be constructed for operation by either hand of a user.

2. Bipolar Pencil with Integrated Switch Body

FIG. 2 shows a conventional Kirwan® disposable bipolar pencil BPE with a tip electrode TE comprising concentric electrodes, as shown by the breakout detail view in FIG. 2. The pencil comprises a tool shaft that houses an inner electrode rod IE surrounded by an insulated tubular outer electrode OE. The tip electrode TE extends from the distal end of a handle portion HP of the pencil shaft. A proximal end of the pencil includes an integral tool plug TP with prongs P1 and P2 that plug into sockets on a power cord receptacle that connects to an electrical generator via a power cord in the fashion depicted in prior art FIG. 1 of the '268 patent vis-à-vis a bipolar forceps.

This arrangement permits standard foot pedal operation, as described above and in the '268 patent in connection with FIG. 1. That is, the surgeon articulates the distal tip of the pencil into the desired position with the electrodes IE and OE in contact with the target tissue, whereby operation of the foot pedal causes an electrical current to pass through the tissue in contact with the exposed ends of the electrodes. Even though this construction provides the advantages of disposability, it still suffers from the shortcomings discussed above inherent in foot pedal operation of an electrosurgical instrument. This is particularly undesirable in this type of instrument, a popular use of which is in delicate ophthalmological procedures that require precise placement of the electrodes.

FIG. 6 improves the conventional bipolar pencil in FIG. 2 by replacing the tool plug with a unilateral switch body 110′ that is configured in a like manner to the switch body 110 described in the text accompanying FIGS. 2-4 of the '036 publication. In the present description and in FIG. 6 a prime (′) symbol denotes features of the switch body that are otherwise identical to features of the switch body 110 in the '036 publication, except that the switch body in FIG. 6 is integrated with the disposable bipolar pencil DBPE. The features of the pencil DBPE that correspond to those of the prior art pencil depicted in FIG. 2 are denoted in FIG. 6 by a prime (′). As in the case of the disposable forceps, the tool portion can be molded in place in the switch body during manufacturing. The power cord 114′, which in turn is integrated with the switch body and thus the tool portion in the same fashion as described above in connection with the disposable forceps embodiment, connects to an electrical generator in like fashion to that described above in connection with FIG. 4 and in the text accompanying FIG. 1 of the '036 publication. Thus, the disposable bipolar pencil forms an integrated pencil/switch body/power cord of unitary construction.

The disposable bipolar pencil DBPE can be operated either by foot pedal, or by moving the actuator lever arm 300 with a finger, as shown in FIGS. 9-12 of the '036 publication. The actuator body 200 and the actuator lever arm 300 are identical to those parts in the '036 publication and in the forceps embedment described above. In addition, the actuator body connects to the switch body 110′ in the same fashion as in the '036 publication and the forceps embodiment, so that the entire subassembly (body 200 and arm 300) can be removed, at which point the bipolar pencil will have the same “feel” to the surgeon as the conventional construction shown in FIG. 2. As in the forceps embodiment described above, the construction of the switch body 100 and the actuator body 200 mounts the actuator lever arm 300 so that it extends toward the pencil's distal end generally along a longitudinal axis so that the lever arm is in position for movement by a finger of the user's hand holding the pencil to close the switch be depressing the switch push button actuator 112′. In summary, the switch body 110′ in effect replaces the molded tool plug TP of the prior art disposable bipolar pencil BPE forceps depicted in FIG. 2, thereby forming an integrated pencil/switch body/power cord of unitary construction.

Since the conventional bipolar pencil BPE is symmetrical about its longitudinal axis, it is operable by either hand. In other words, although FIG. 6 depicts the present embodiment in a right-handed orientation, rotating the entire tool 180° about its longitudinal axis will place the lever arm 300 on the pencil's left side. In some configurations the pencil may have a finger guide FG′ near its distal end. The present construction could include such a guide on the opposite side (“underneath” as seen in FIG. 6) for left-hand operation, In another variation, the unilateral switch body 110′ could instead have a bilateral construction like the switch body 1110 shown in FIGS. 4 and 5 to allow for left-handed operation. In still another variation, the handle portion HP′ can be integrally mounted to the switch body 110′ for rotation about the longitudinal axis of the handle portion to selectively locate the actuator lever arm 300 at a desired angular position. In that case, the rotational mounting could comprise one or more detents to hold the actuator lever arm in one or more preset positions. For example, one such position could be as shown in FIG. 6 for right-hand operation, another could be rotated 180° for left-hand operation. Accordingly to user preference, the switch body and the tool could have a detent at 90° rotation if a user desired to use the pencil in such an orientation, or wished to use it with a pistol-type actuator. It will be appreciated that the tool portion of the disposable forceps described above, and the disposable probe described in the next section can also adopt this construction.

3. Bipolar Probe with Integrated Switch Body

FIG. 3 shows a conventional Kirwan® disposable bipolar probe BPR with an insulated probe arm PA that terminates in a tip electrode TE extending from the distal end of a handle portion HP. The tip electrode TE comprises two closely spaced electrodes E1 and E2 as shown by the breakout detail view in FIG. 3. The electrodes are insulated from each other and are typically about 4 mm apart to allow for very precise application of electrical current to the target tissues. A proximal end of the probe includes an integral tool plug TP with prongs P1, P2 that plug into sockets on a power cord receptacle that connects to an electrical generator via a power cord in the same fashion depicted in FIG. 1 of the '268 patent.

This arrangement permits standard foot pedal operation, as described above and in the '268 patent in connection with FIG. 1. That is, the surgeon articulates the electrodes E1 and E2 into the desired position across the target tissue, whereby operation of the foot pedal causes an electrical current to pass through the tissue in contact with the electrodes. This construction, which is also used in many delicate procedures, suffers from all of the shortcomings discussed above inherent in foot pedal operation of an electrosurgical instrument.

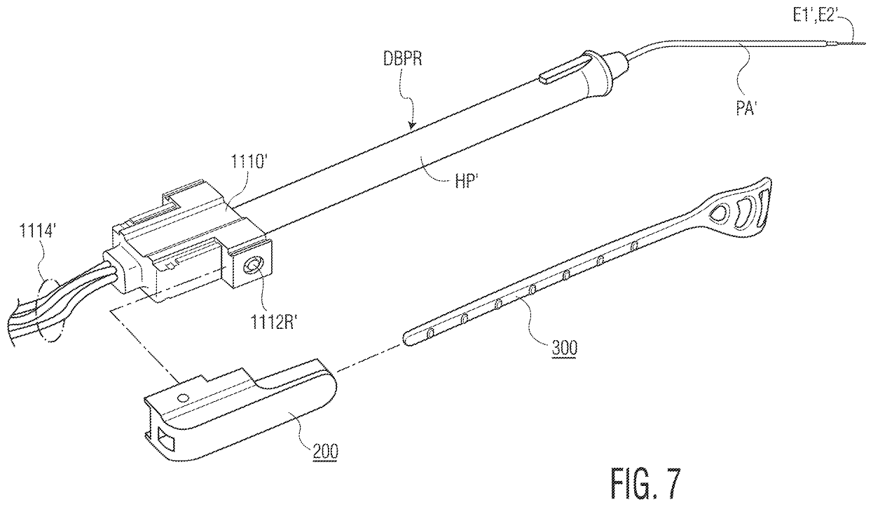

FIG. 7 improves the conventional bipolar probe in FIG. 3 by replacing the tool plug with a bilateral switch body 1110′ that is configured in a like manner to the switch body 1110 described in connection with the forceps embodiment described above in connection with FIGS. 4 and 5. In the present description and in FIG. 7 a prime (′) symbol denotes features of the switch body that are otherwise identical to features of the switch body 1110 integrated with the disposable forceps DFC as described above. The prior art probe BPR, like the forceps FC and the pencil BPE, can be used by either hand without modification. However, the disposable constructions described herein, particularly the disposable forceps DFC and disposable probe DBPR have distinct left- and right-hand orientations when the lever arms 300 are in place. That is, the distal electrodes E1′ and E2′ are oriented relative to the handle portions HP′ such that manipulation of the instruments and by the same hand used to operate the lever arm requires the switch body to accommodate operation by either hand. The term “bilateral switch body” used herein means that the switch body includes right- and left- hand connecting structure that enables the actuator body 200 to connect to the switch body at two places. The right- and left-hand connecting structures (donated by the letters “R” and “L” in FIG. 5) will typically be on opposite sides, 180° apart relative to the longitudinal axis of the handle portion, but the connecting structure in that respect can be any configuration that accommodates manipulation of the instrument into position and operation of the lever arm using the same hand.

As in the forceps and pencil embodiments, the tool shaft with the electrodes E1′ and E2′ comprises a tool portion that is integrally mounted to the switch body 1110′ to form a unitary structure. The power cord 1114′, which in turn is integrated with the switch body and thus the tool portion, connects to an electrical generator in like fashion to that described above in connection with the previous embodiments. Thus, the disposable bipolar probe DBPR can be operated either by foot pedal, or by moving the actuator lever arm 300 with a finger, as shown in FIGS. 9-12 of the '036 publication. The actuator body 200 and the actuator lever arm 300 are identical to those parts in the '036 publication. In addition, the actuator body connects to the switch body 1110′ in the same fashion as in the '036 publication, so that the entire subassembly (body 200 and arm 300) can be removed, at which point the bipolar probe will have the same “feel” to the surgeon as the conventional construction shown in FIG. 2. In like fashion to the forceps and probe embodiments described above, the construction of the switch body 1110′ and the actuator body 200 mounts the actuator lever arm 300 so that it extends toward the probe's distal end generally along a longitudinal axis so that the lever arm is in position for movement by a finger of the user's hand (either right or left, as the case may be) holding the probe to close the switch be depressing the switch push button actuator 1112R′.

### D. Alternate Constructions and Applications

The particular constructions described above are meant as exemplary embodiments of applications of the concepts described herein. For example, an alternate construction could employ wireless communication instead of wired leads to the electrical generator. In another variation the various types of switch bodies described herein could include a battery powered Bluetooth® transceiver that communicates with a paired Bluetooth® transceiver in the electrical generator. In yet another embodiment, the switch body could include batteries sufficiently powerful to provide operating current to the forceps electrodes, thus eliminating the need for a separate electrical generator. It will be further understood that certain constructional details have been included solely to provide examples of ways in which the instruments described herein can be realized. The constructional features of molding the instrument tool portions with the switch body during manufacture and using a molded sealing collar around the power cord/switch body connection are examples of such features that can be realized in any way that provides the same effect.

It will be further understood that the arrangements disclosed above, applied to various types of disposable instruments to enable single-finger actuation, can also be applied to other disposable instruments as well. For example, the principles governing the construction of the switch body and actuator assembly could also be equally applied to a monopolar pencil or a monopolar probe with a handle portion and a single tip electrode.

The foregoing description has been presented solely for purposes of illustration and description of the subject matter covered by the claims that follow. It is not intended to be exhaustive or to limit the claimed subject matter to the precise form disclosed, and other modifications and variations may be possible in light of the above description. Those skilled in the art will readily recognize that only selected embodiments of the disclosed subject matter have been depicted and described, and it will be understood that various changes and modifications can be made other than those specifically mentioned without departing from the spirit and scope of the subject matter herein. Specific embodiments were chosen and described solely in order to best explain principles underlying the disclosed subject matter and its practical applications, thereby to enable those skilled in the art to best utilize it in its various embodiments and various modifications as are suited to the particular use contemplated. It is intended that the appended claims be construed to include other alternative embodiments except insofar as limited by the prior art.

What is claimed is:

1. A disposable electrosurgical instrument comprising a tool portion, a switch body, and a power cord integrated into a unitary construction, wherein:
said tool portion extends from a proximal end to a distal end with a handle portion therebetween for manipulation of the tool by the hand of a user holding the tool, wherein the tool portion includes at least one electrode at the distal end for applying to tissue electrical current introduced to the tool portion when the user manipulates the electrode into contact with the tissue;
said switch body is integrally mounted to the tool portion at the proximal end thereof, wherein the switch body includes (i) a switch electrically connected to the electrode, the switch being movable between an open position and a closed position, and (ii) connecting structure for removably connecting to the switch body an actuator body for mounting an actuator arm in position for movement by the user's hand holding the tool to place the switch in the closed position; and
said power cord is integrally mounted to the switch body, said power cord being adapted to place the switch in electrical contact with an electrical generating apparatus to introduce electrical current to the electrode when the switch is in the closed position.

2. A disposable electrosurgical instrument as in claim 1, wherein said tool portion includes two electrodes at the distal end thereof for providing a current path through tissue in contact with both electrodes when the switch is in the closed position.

3. A disposable electrosurgical instrument as in claim 1, wherein said actuator arm comprises an actuator lever arm positioned for movement by a finger of the user's hand holding the tool to place the switch in the closed position.

4. A disposable electrosurgical instrument as in claim 3, wherein:
when the actuator body is mounted to the switch body, the tool portion is configured for manipulation of the at least one electrode into contact with the tissue by one of the user's right hand or left hand and movement of the lever arm by a finger of the same hand; and
the switch body connecting structure includes right-hand connecting structure for mounting the actuator body on the switch body for movement by a finger of the user manipulating the tool portion with the right hand and left-hand connecting structure for movement by a finger of the user manipulating the tool portion with the left hand.

5. A disposable electrosurgical instrument as in claim 4, wherein said tool portion comprises a forceps including two tines extending to the distal end from the proximal end, wherein each tine has an electrode at the distal end thereof insulated from each other, and the actuator lever arm extends toward the distal end of the forceps generally in the direction of a longitudinal axis thereof when the actuator body is connected to the switch body.

6. A disposable electrosurgical instrument as in claim 4, wherein said tool portion comprises a bipolar probe comprising a tool shaft extending to the distal end from the proximal end, the distal end including a first tip electrode and a second tip electrode spaced from the first tip electrode and insulated therefrom, and the actuator lever arm extends toward the distal end of the tool shaft generally in the direction of a longitudinal axis thereof when the actuator body is connected to the switch body.

7. A disposable electrosurgical instrument as in claim 3, wherein said tool portion comprises a bipolar pencil comprising a tool shaft extending to the distal end from the proximal end, the distal end including an inner electrode and an outer electrode surrounding the inner electrode and insulated therefrom, and the actuator lever arm extends toward the distal end of the tool shaft generally in the direction of a longitudinal axis thereof when the actuator body is connected to the switch body.

8. A disposable electrosurgical instrument comprising:
a unitary construction including (a) a tool portion extending from a proximal end to a distal end with a handle portion therebetween for manipulation of the tool by the hand of a user holding the tool, wherein the tool portion includes at least one electrode at the distal end for applying to tissue electrical current introduced to the tool portion when the user manipulates the electrode into contact with the tissue, (b) a switch body integrally mounted to the tool portion at the proximal end thereof, said switch body including a switch electrically connected to the electrode and being movable between an open position and a closed position, and (c) a power cord integrally mounted to the switch body, said power cord being adapted to place the switch in electrical contact with an electrical generating apparatus to introduce electrical current to the electrode when the switch is in the closed position; and
an actuator body having actuator body connecting structure for mounting said actuator body to cooperating switch body connecting structure, said actuator body including an actuating arm movably mounted to said switch body, wherein said actuating arm is adapted to removably mount an actuator lever arm in position for movement by a finger of the user's hand holding the instrument to move the actuating arm to place the switch in the closed position.

9. A disposable electrosurgical instrument as in claim 8, wherein said actuating arm comprises an actuator pivot arm rotatably mounted to said actuator body.

10. A disposable surgical instrument as in claim 8 wherein said actuator pivot arm and said actuator lever arm include cooperating positioning means for releasably holding said actuator lever arm in a plurality of positions extending for different lengths along a longitudinal axis of said tool portion.

11. A disposable electrosurgical instrument as in claim 8, wherein said tool portion includes two electrodes at the distal end thereof for providing a current path through tissue in contact with both electrodes when the switch is in the closed position.

12. A disposable electrosurgical instrument as in claim 11, wherein said tool portion comprises a bipolar pencil comprising a tool shaft extending to the distal end from the proximal end, the distal end including an inner electrode and an outer electrode surrounding the inner electrode and insulated therefrom, and the actuator lever arm extends toward the distal end of the tool shaft generally in the direction of a longitudinal axis thereof when the actuator body is connected to the switch body.

13. A disposable electrosurgical instrument as in claim 8, wherein:
when the actuator body is mounted to the switch body, the tool portion is configured for manipulation of the at least one electrode into contact with the tissue by one of the user's right hand or left hand and movement of the lever arm by a finger of the same hand; and
the switch body connecting structure includes right-hand connecting structure for mounting the actuator body on the switch body for movement by a finger of the user manipulating the tool portion with the right hand and left-hand connecting structure for movement by a finger of the user manipulating the tool portion with the left hand.

14. A disposable electrosurgical instrument as in claim 13, wherein said tool portion comprises a forceps including two tines extending to the distal end from the proximal end, wherein each tine has an electrode at the distal end thereof insulated from each other, and the actuator lever arm extends toward the distal end of the forceps generally in the direction of a longitudinal axis thereof when the actuator body is connected to the switch body.

15. A disposable electrosurgical instrument as in claim 13, wherein said tool portion comprises a bipolar probe comprising a tool shaft extending to the distal end from the proximal end, the distal end inkling a first tip electrode and a second tip electrode spaced from the first tip electrode and insulated therefrom, and the actuator lever arm extends toward the distal end of the tool shaft generally in the direction of a longitudinal axis thereof when the actuator body is connected to the switch body.

16. A disposable electrosurgical instrument comprising a tool portion, a switch body, and a power cord integrated into a unitary construction, wherein:
said tool portion extends from a proximal end to a distal end with a handle portion therebetween for manipulation of the tool by the hand of a user holding the tool, wherein the tool portion includes at least one electrode at the distal end for applying to tissue electrical current introduced to the tool portion when the user manipulates the electrode into contact with the tissue;
said switch body is integrally mounted to the tool portion at the proximal end thereof, wherein the switch body includes (i) a switch electrically connected to the electrode, the switch being movable between an open position and a closed position, and (ii) an actuator body integral with said switch body for mounting an actuator lever arm in position for movement by a finger of the user's hand holding the tool to place the switch in the closed position; and
said power cord is integrally mounted to the switch body, said power cord being adapted to place the switch in electrical contact with an electrical generating apparatus to introduce electrical current to the electrode when the switch is in the closed position.

17. A disposable electrosurgical instrument as in claim 16, wherein said actuator body comprises an actuating pivot arm rotatably mounted to said actuator body.

18. A disposable electrosurgical instrument as in claim 17, wherein said actuating pivot arm and said actuator lever arm include cooperating positioning means for releasably holding said actuator lever arm in a plurality of positions extending for different lengths along a longitudinal axis of said tool portion.

19. A disposable electrosurgical instrument as in claim 16, wherein said tool portion includes two electrodes at the distal end thereof for providing a current path through tissue in contact with both electrodes when the switch is in the closed position.

20. A disposable electrosurgical instrument as in claim 16, wherein:
the tool portion is configured for manipulation of the at least one electrode into contact with the tissue by one of the user's right hand or left hand and movement of the actuator lever arm by a finger of the same hand; and
the switch body includes one of (a) a right-hand actuator body disposed for movement by a finger of the user manipulating the tool portion with the right hand, or (b) a left-hand actuator body disposed for movement by a finger of the user manipulating the tool portion with the left hand.
